(19) United States
(12) Patent Application Publication
(10) Pub. No.: US 2015/0237902 A1
(43) Pub. Date: Aug. 27, 2015

(54) INFANT FORMULA FOR OPTIMAL GROWTH, GASTROINTESTINAL PROTECTION AND IMMUNOLOGICAL PROTECTION OF INFANTS

(71) Applicant: NUCITEC, S.A. DE C.V., Querétaro, Quéretaro (MX)

(72) Inventors: Jorge Luis Rosado Loria, Queretaro (MX); Miguel Angel Duarte Vázquez, Queretaro (MX)

(73) Assignee: Nucitec, S.A. DE C.V., Querétaro, Quéretaro (MX)

(21) Appl. No.: 14/427,379

(22) PCT Filed: May 2, 2013
(86) PCT No.: PCT/MX2013/000057

§ 371 Date: Mar. 11, 2015

(30) Foreign Application Priority Data
Sep. 11, 2012 (MX) MX/A/2012/010488

(51) Int. Cl. A23L 1/29; A61K 31/7016; A61K 38/17; A61K 31/20; A61K 31/702; A61K 31/202; A61K 31/7076; A61K 31/708; A61K 31/7068; A61K 31/7072; A61K 45/06; A61K 31/047; A61K 31/14; A61K 31/185; A61K 31/205; A23L 1/305; A23L 1/30; A23L 1/302; A23L 1/303; A23L 1/304; A61K 38/38

(52) U.S. Cl. CPC A23L 1/296; A61K 38/38; A61K 31/7016; A61K 38/1709; A61K 31/20; A61K 31/702; A61K 31/202; A61K 31/7076; A61K 31/708; A61K 31/7068; A61K 31/7072; A61K 45/06; A61K 31/047; A61K 31/14; A61K 31/185; A61K 31/205; A23L 1/3056; A23L 1/3012; A23L 1/3008; A23L 1/30; A23L 1/302; A23L 1/303; A23L 1/304; A23V 2002/00

(57) ABSTRACT

An infant formula specially designed for covering nutrition necessities of infants between 0 and 36 months of life is described, which reduces the intolerance problems related to the consumption of infant formulas currently found in the art.

### FIELD OF THE INVENTION

The present disclosure belongs to the fields of nutrition and infant formulas. More specifically, the disclosure is related with the obtaining of a composition to be used as a feeding formula for children between 0 and 36 months of age.

### BACKGROUND OF THE DISCLOSURE

Breastfeeding is the optimal way of feeding for the newborn. In many occasions, it is not possible due to different reasons, mainly because the mother works or because there is some physiological impediment that avoids the child to be fed from her mother breast. In these cases, the baby must be fed with an infant formula made based on the different components of the cow's milk. The term infant formula is employed to name products for infants feeding, which pretend to completely or partially substitute human milk to cover all the needs of the newborn feeding.

According to the 2006 National Nutrition Survey, 75% of the infants less than 4 months old were partially or completely fed with breast milk substitutes or with infant formulas. Usually these formulas are well accepted, but many children in their first year of life, from 4 to 5% according to some authors (Bock S A. Prospective appraisal of complaints of adverse reactions to foods in children during the first three years of life. Pediatric 1987; 79:683-688.), or from 2 to 5% according others (Host A, Halken S A. A prospective study of cow's milk allergy in Danish infants during the first three years of life. Clinical course in relation to clinical and immunological type of hypersensitivity reaction. Allergy 1990; 45:587-596; Schrander J J P, Van Den Bogart J P H, Forget P P et als. Cow's milk protein intolerance in infants under 1 year of age: a prospective epidemiological study. Eur J Pediatr 1993; 152: 640-644) suffer some kind of gastrointestinal disorder by using these formulas. Among the most common gastrointestinal disorders presented there is reflux and/or regurgitation, colic, appetite decrease, mainly due to the intolerance to the components of the formula.

There are two types of formulas according to the age of the infant: starting formulas and follow up formulas. The reason for recommending two different types of formulas in the first year of life is that at 6 months of age the infant reaches the maturity in the digestion and intestinal absorption processes, as well as in the activity of the enzymes of the intermediary metabolism and the renal excretory function.

In spite of, there are certain differences between the human milk and the current infant formulas. One of the main differences is the composition and concentration of protein, human mature milk provides approximately 10-12 g/L of total protein (Feng et al., 2009) and is rich in essential amino acids. The standard infant formulas are made from cow's milk, which has a higher protein concentration and a different amino acids profile than the human milk. The higher protein concentration (14-15 g/L) is necessary in order to provide enough quantity of essential amino acids.

It is believed that the higher concentration of protein in the infant formulas compared to the human milk is responsible of the difference in growing observed between them. The newborns fed with infant formulas present a higher concentration of branched chain amino acids in blood than newborns fed with human milk (Tikanoja and Simell, 1983; akeson et al., 1988). The rise in the plasmatic concentration of these amino acids has been positively related to an increase in the insulin release, which leads to an increase in the recapture of cellular glucose and lipolysis inhibition. It has been proposed that these metabolic disorders are responsible for greater weight gain and a greater body mass index in infants fed with infant formula than in breast milk fed infants.

These metabolic changes in early ages have been identified as protection factors that reduce the probability of developing obesity and overweight in posterior stages of life (Taveras et al., 2009).

On the other hand, human milk and infant formulas have an important difference about the ratio of whey proteins and casein proteins. Originally, infant formulas were based in cow's milk without further modifications and thus they had a protein profile that comprised 20% of whey proteins and 80% of casein proteins. Compared to the static nature of the cow's milk, human milk is a dynamic fluid, which changes from a high ratio whey:casein (more than 80% of casein protein) in the beginning of lactation, to a ratio 60:40 in mature milk, reaching a ratio 50:50 in the final stages of lactation (Rudloff y Kunz, 1997). As a consequence, the ratio whey:casein in the infant formulas has been adjusted to be the closest to human milk (approximately 60:40) by the addition of whey protein concentrate. The first whey-dominant formula was developed in 1961, wherein the protein profile was closest to the human milk. These whey-dominant formulas gave as a result a minor protein and minerals concentration than that found in the casein-dominant formulas giving as a result formulas with a lower solute load to the kidney.

Despite the efforts that have been made to try to match the infant formulas to the breast milk composition and achieve the benefits of these in the babies, there are still significant differences. One important difference is that human milk has a considerably higher concentration of alphalactalbumin than cow's milk or than the milk whey-added formulas. Both in the cow's milk and the whey-dominant formulas predominates the beta lactoglobulin. The beta lactoglobulin is not expressed by the mammary gland so it is not found in the human milk, and it is highly antigenic so its presence in whey-dominant formulas is related to a higher incidence of allergy to the cow's milk protein.

However, the most important difference between the proteins found in human milk and those currently used in infant formulas is the type of casein. Caseins are the main component of the cow's milk protein and thus of the infant formulas and are the main source of amino acids for the newborn. Three important subgroups of casein have been identified; alpha casein, beta casein and kappa casein, being the main difference in the beta casein fractions. The beta casein is one of the most abundant proteins of the milk casein, being around 30% of the total cow's milk. There are two main forms of beta casein; these two forms are known as beta casein A1 and A2. The difference between casein A1 and A2 is one single amino acid in position 67, while A1 has histidine, A2 has proline in the same position. This difference of one amino acid affects the proteolytic process of the beta casein in the mammalian intestine. In vitro studies have shown that the bioactive peptide beta casomorphin 7 (BCM7) is generated by the gastrointestinal proteolytic digestion of beta casein A1, but not of A2, giving as a result different levels of bioactive peptides produced by the two principal types of beta casein variants. The BMC7 has a strong opioid activity, so the milk that does not contain beta casein A1 must not have an opioid action. Other characteristics of the BMC7 are the following: it promotes LDL oxidation which is associated with a higher risk of heart disease, affects the insulin regulation, causes histamine release in humans as in the allergenic responses, affects (decreases) the immune human response, is related with type 1 diabetes which is developed in the childhood, directly acts in the intestine coating affecting the digestive process, might overpass the hematoencephalic barrier and affect regions of the brain producing thus schizophrenia and autism.

A huge number of publications support the epidemiological evidence that suggest that excessive intake of β-casein A1 is detrimental for human health. For example, the patent WO 96/14577 describes the impact of β-casein A1 on type 1 diabetes. The epidemiological evidence suggests that β-casein A1 stimulates the diabetogenic activity in human. Besides, the document WO 96/14577 describes the induction of autoimmune diabetes, or type 1, in a mice model for β-casein A1 consumption. The disclosure described in the mentioned patent is centered in reducing the risk of developing type 1 diabetes in a susceptible individual, restricting milk ingestion or dairy products that contain β-casein A1. The epidemiological evidence establishes a correlation between the β-casein A1 consumption in diverse populations and the coronary heart disease incidence.

Therefore, the link between the β-casein A1 and diabetes and the relationship between β-casein and the coronary heart disease have been documented. Furthermore, it has been recently shown that the deleterious effects of β-casein A1 may origin neurological disorders. There is an important difference between the beta casein protein of the human milk and the beta casein produced by cow's. The beta casein in human milk is closest to the type A2, which means that in general human milk produces much less beta casomorphin than the one produced when cow's milk is consumed and thus the children fed with infant formulas made from cow's milk are going to produce more beta casomorphin than children fed with human milk.

Lactoferrin is one of the most important components of the human milk proteins, it constitutes between 10-15% of the total protein, but it is found almost absent in cow's milk. The lactoferrin is a glycoprotein of molecular weight close to 80,000 Da which is able to bind two iron atoms. Due to this iron-binding capability, functions in the iron-nutritional state maintaining have been attributed to lactoferrin and also bacterial growth inhibitory properties, so its presence in infant formulas is completely justified.

Recent studies suggest that the nucleotides of the diet (breast milk) might be imperative for the newborn. Tissues in fast development as the intestinal epithelium or the lymphoid cells need an increased nucleotides quantity. These nucleotides form part of the non-protein nitrogen fraction of the breast milk that constitute between 18-30% of the total nitrogen, while in the cow's milk they only constitute the 5%.

Another fraction of the cow's milk solids that must be modified in order to obtain infant formulas as adequate as possible to the digestive apparatus of the newborn is fat. The fat composition (4.4-6.0 g/100 kcal) must be the one that obtains an 85% of absorption. This is difficult to obtain with the cow's milk fat due to its high content of saturated fatty acids (of more difficult digestion). The breast milk lipids have a 70% of the fatty acids in beta position, higher than the usual in the infant formulas used nowadays.

Human milk contains lactose and other carbohydrates such as oligosaccharides. It is calculated that human milk contains around 12 g/L of oligosaccharides from which more than 100 different structures have been identified corresponding to different oligosaccharides. Some of these oligosaccharides are not digested in the gastrointestinal tract, so they constitute “soluble fiber” of the breast milk, such in a way that it provides the substrate for the bacteria in the colon of the infant. It has been observed that children fed with breast milk have a higher and better-quality intestinal flora concentration than those fed with infant formulas in which oligosaccharides are not present. Intestinal flora is a part of a complex ecosystem, which has a great influence on the postnatal development of the immune system, so this flora stimulation with prebiotic oligosaccharides may be an effective method to alter the immune system.

In the market there are some infant formulas with one or more of the characteristics mentioned above that provide a nutritional option for infants less than 6 months of age, other that are adequate in order to maintain the growth of the infant at this age and until 36 months old but that, nevertheless, present some disadvantages from the nutritional point of view or that due to its composition have the risk of developing metabolic disorders in later stages of development.

This way, infant formulas have been developed, each of them with its own limitations. The patent solitude US 2007/0031537 A1 describes a formula intended for infants, which composition includes as a protein source the combination whey:casein of 60:40, from a higher range of even 20:80, which lacks specificity, resulting a very limited proposal to generate more benefits to the newborn. Besides, it does not mention addition of alpha lactalbumin, so it is possible that when increasing the quantity of whey protein in this formula the quantity of whey beta lactalbumin is also increasing, which has a great antigenic potential. In the same formula it is not considered the prebiotic (oligosaccharides) addition, it also provides adequate quantities of arachidonic acid (ARA) and docosahexaenoic acid (DHA), however, the formula does not consider the addition of a fat source with palmitic acid in beta position (beta carbon). Furthermore, the probiotics presence, far from an advantage, represents a disadvantage, mainly in less than 6 months old children, since at this age the digestive system of the baby presents certain immaturity level, with some fissures in the intestinal wall, which could provoke the probiotic escapes into the bloodstream.

In the patent document US 2011/0244072 A1 it is described an infant formula and a body fat accumulation reduction method in infants by administrating this formula. The document does not describe alpha lactalbumin enrichment, so due to the fact of having only whey protein it contents high quantities of the highly allergenic beta lactoglobulin protein. In the same way it is not considered the beta casein A1 substitution for beta casein A2. The fat source considered in the disclosure is palm oil, rich in palmitic acid in position 1 and 3, it is not considered the addition of prebiotics or nucleotides, which represents a clear disadvantage.

In the patent US 2007/0110849 A1, there is described a formulation that can be used as first infant formulas or follow-up formulas, however in this composition, despite the high whey protein levels that are present, enrichment with alpha lactalbumin is not mentioned, which indicates that beta lactoglobulin concentrations (highly allergenic), which represent the main fraction of whey proteins are increased automatically in the formula. Beta casein A1 substitution for beta casein A2 is neither mentioned and neither the addition of lactoferrin, it does not consider the addition of palmitic acid in beta position, so there is constipation risk. The formula does not consider prebiotics (oligosaccharides) addition but it does consider probiotics, which are not recommendable in less than 6 months old children.

In the patent US 2009/017579 A1 there is described a method for reducing the side effects incidence in infants in the long term, this method consists in administrating a nutritional formula which provides less than 1 g of protein for every 100 mL of formulation and less than 50 kcal per every 100 mL of the formula.

In the document of the patent US 2011/0195144 A1, there is described a disclosure related to the use of a protein source, either whey or casein to provide a nutrition system for infants, this system consists in two adapted formulas, each of them for a different age of the infant. Each of the formulas comprises a protein source where the ratio whey protein:casein is found in the range from 100:0 to 40:60, and the protein content of each formula is found in the range between 1.5 to 3.0 g of protein/100 kcal. The range of the ratio whey:casein represent a disadvantage, it is mentioned that the source of protein is not important and that alpha lactalbumin can be used in combination with beta lactoglobulin.

In consequence, it is possible achieve a reduction of beta casein A1 in milk or in infant formulas derived from it, which will reduce production of BCM7, the risk of suffering certain disorders related to high production of BCM7 will be reduced. In the same way it is important to achieve a fatty acids similar profile to that of breast milk and that includes fatty acids in beta position in a way that reduces the calcium soaps formation in order to diminish constipation problems due to stool hardening. Due to the fact that the addition of lipids with the palmitic acid in beta position (beta palmitate) makes the stool softer and favors the lipids and divalent metals such as magnesium and calcium absorption. Due to the previously mentioned, a reduction of the constipation events, which are very common in children fed with infant formulas, is achieved.

Whenever nucleotides are non-protein nitrogenous compounds, which content in breast milk is between 2 and 5% of the total non-protein nitrogen content, besides that nucleotides are non-protein nitrogenous compounds that are part of the DNA and RNA structure and thus, are imperative during the growth and also participate in various biochemical processes of the cellular metabolism specially in the protein synthesis, thus it is expected that infant children fed with infant formulas which comprise nucleotides show less infectious diarrhea incidence, a better immunologic response to vaccines and an improvement in the growth of low weight newborns.

Therefore, it is expected the development of supplemented infant formulas for infants feeding which composition resembles breast milk. These formulas would provide a better nutritional balance, having a higher tolerance and reducing in an important grade the side effects produced by other formulas in the short and in the long term.

### SUMMARY OF THE INVENTION

The present disclosure provides a supplemented infant formula which provides a protein quantity of 1.8 g/dl, in a whey:casein ratio of 65:35 and is enriched with alpha lactalbumin, which forms 26% of the total protein. The composition of the disclosure is also characterized for having a higher percentage of beta casein A2 similarly to the breast milk. The formula of the disclosure is enriched with prebiotics (fructooligosaccharides and galactooligosaccharides), docosahexaenoic acid (DHA), arachidonic acid (ARA), palmitic acid in beta position and nucleotides.

The disclosure represents a novelty due to the modification of the nutritional components of cow's milk to resemble as possible to breast milk, diminishing in this way the adverse events produced by cow's milk consumption in short and long term and giving guarantee of the optimal growth of the infant.

In the current disclosure an infant formula was added with bovine alpha lactalbumin with the objective of reaching an amino acids profile in infant's plasma similar to that which could be found with human milk consumption, besides reducing beta lactoglobulin concentration and thus reducing the possibility of a consequent allergy to cow's milk protein. Amino acids composition present in alpha lactalbumin is well balanced, with a high ratio of certain essential amino acids (lysine, leucine, tryptophan, cysteine), so the increase in concentration of alpha lactalbumin in the formula will allow to reduce total protein concentration with a lower renal load for the infant whose excretion system is immature during the first months of life.

Besides being a good essential amino acids source, alpha lactalbumin has diverse positive physiological effects such as, antimicrobial activity, increase in the immune function, prebiotic function, and increase in minerals absorption. This beneficially affects the nutrition state, reduce the possibility of diseases and increase the health and the development of the child.

### DESCRIPTION OF THE DISCLOSURE

The main disadvantage of the formulas found in the previous art is that they have been designed based on cow's milk protein which has significant differences with respect to breast milk, as the whey:casein ratio. In those in which this ratio has been tried to be corrected, the content of whey has been indiscriminately increased, without taking into account that the content of beta lactoglobulin also increases automatically due to the fact that it is the main protein of whey and it is highly allergenic. Due to this fact, allergenic reactions when consuming infant formulas, has increased in a very important manner, with an incidence between 5 and 7% in children under one year of age (Sánchez et al., 2007. Revista de Enfermedades Infecciosas en Pediatria; 21:51-59). Furthermore, addition of alpha lactalbumin, which in breast milk forms even 29% of the total protein, has not been considered in the infant formulas where whey fraction has been increased.

On the other hand, none of the existing formulas in previous art has substitute beta casein A1 for beta casein A2, so another disadvantage of these formulas is the high risk for the consumer of suffering metabolic disorders or central nervous system disorders due to BCM7 release during beta casein A1 hydrolysis.

Additionally, a careful analysis of the current commercialized formulas shows that the nutritional profile of these products is found limited in some nutrients which are imperative for an adequate physical and mental development of the infant such as: DHA (docosahexaenoic acid) for the brain development, ARA (arachidonic acid) for the cornea development, linolenic acid which is DHA promoter, taurine which is an essential amino acid in the first 6 months of life, nucleotides that positively interfere in the fat metabolism and the intestinal function and their deficiency may have undesirable effects for the child; so another advantage of the formula of the disclosure is its nutrients composition since it was specially designed to emulate as much as possible breast milk and this way guarantee the adequate infant development.

Beta casein is one of the main proteins of casein in milk, accounting for about 30% of the total protein of cow's milk. Two isoforms have been identified according to their structural difference, which are A1 and A2, this structural difference affects the proteolytic process. When structure A1 is more abundant in milk the bioactive peptide beta casomorphin which is involved in the tendency to promote LDL oxidation, heart disease, affects insulin formation regulation, increases the allergenicity and reduces the digestibility process capability is produced.

A prebiotic is a non-digestible ingredient that positively affects the host, through a selective stimulation of grow and activity of certain bacteria in the colon, improving the health state of who is consuming it. Prebiotics examples include different oligosaccharides, such as fructooligosaccharides (FOS) and galactooligosaccharides (GOS).

Nucleotides are non-protein nitrogenous compounds, which content in breast milk is between 2 and 5% of the non-protein total nitrogen. Cow's milk has a lower content of nucleotides than human milk and its nucleotides profile is significantly different. Nucleotides are non-protein nitrogenous compounds that form part of the structure of DNA and RNA, and thus, are imperative during the growth, besides they participate in different biochemical process of the cellular metabolism specially protein synthesis. Breast milk provides nucleotides and is believed that in fast-growth periods this exogenous nucleotides contribution may suppose a metabolic saving to its endogenous production and favor immunity and growth. Studies in infant children fed with nucleotides supplemented infant formulas show less incidence of infectious diarrhea, a better immunologic response to vaccines and an improvement in the growth of low weight newborns.

### OBJECTIVE OF THE DISCLOSURE

The objective of the present disclosure is to provide a nutritional formula for using as exclusive feeding in infants from zero to 36 months of age that is similar to breast milk.

A second objective is to provide a feeding formula for infants from 0 to 36 months, which overcome the disadvantages and the negative effects that currently occur with the use of formulas for infants feeding, besides being an excellent alternative to support the optimum growth and development of infants.

Another objective of the disclosure is to provide a unique combination of nutrients which assure that children fed with the infant formula object of this disclosure have the growth and metabolic patterns similar to those in infants fed with breast milk, in an attempt of reducing the health risks that might be produced in the late childhood and adulthood with the consumption of the currently available infant formulas.

### SPECIFICATION OF THE DISCLOSURE

The protein supply of the formula of the present disclosure is approximately 1.8 g/dL since this is the closest quantity to that of breast milk; current infant formulas have between 2.4 and 2.8 g/dL which causes a metabolic effort to the kidney which is the organ by which urea product of proteins metabolism is excreted.

The composition of the disclosure is characterized for having a greater percentage of beta casein A2 than normal infant formulas. In normal milk from the total of beta casein, no more than 30% is found as A2; while in the present formula more than 60% of beta casein is found as beta casein A2.

The present formula has a whey:casein ratio of 65:35 that corresponds to the same ratio present in breast milk in the first semester of life, which guarantees an adequate provision of essential amino acids. The ratio between the casein and the milk whey protein is usually found in infant formulas as 80:20, although formulas with a higher content of whey have been developed recently reaching a casein:whey ratio of 40:60. With this composition milk intolerance is significantly reduced and also the side effects such as colic, constipation and reflux that continuously are presented with the current formulas are reduced.

In the disclosure composition, besides the increase of whey proteins concentration, there is alpha lactalbumin enrichment, in a ratio between 20 and 30% of the total protein, differently from the existing formulas which only have between 11 and 16%.

The infant formula of the present disclosure is supplemented with the bioactive peptide lactoferrin. Lactoferrin promotes the development and maturation of the gastrointestinal tract in the newborn, besides it has a positive effect in the iron absorption and has antimicrobial properties that diminish the probability of acquiring infections. Lactoferrin content in the formula of the present disclosure is between 0.005 g and 1.0 g/100 g of formula.

The protein sources used are isolate whey proteins concentrate, alpha lactalbumin, lactoferrin and casein, obtaining a casein:whey milk proteins ratio of 35:65, and a proportion of beta casein A2 of more than 2% of the total beta caseins. The used protein sources provide approximately 26% of the total protein as alpha lactalbumin and less than 7% as beta lactoglobulin.

Carbohydrates in the formula of the disclosure are present in concentrations between 9-14 g/100 kcal. The chosen carbohydrate is lactose. Lactose is the naturally present carbohydrate in milk and it is very important to favor calcium and other minerals absorption and retention. The calcium absorption in formulas which comprise lactose as carbohydrate is 10% higher than in formulas which substitute it for glucose polymers.

Carbohydrates of the formula supply 46% of total energy and are exclusively formed by lactose and a mixture of oligosaccharides.

The formula of the present disclosure is enriched with prebiotics, such as the fructooligosaccharides (FOS) and galactooligosaccharides (GOS). These oligosaccharides form the third main component of breast milk and are considered responsible for the great quantity of bifidogenous gastrointestinal flora of breastfed infants. A rise in bifidogenous flora is usually correlated to an increase in infections resistance and a reduction in the risk of suffering atopic dermatitis. In the disclosure composition the sum of both oligosaccharides is found to be between 0.3 and 0.8 g/100 ml of reconstituted formula, in a combination of 10% FOS and 90% GOS.

Also, these oligosaccharides are considered soluble dietary fiber and increase fecal mass in a dose-dependent way which avoids constipation and reduces the risk of the infants of suffering abdominal colic.

The fats in the present disclosure provide between 30 and 55% of the total energy. In the formula of the disclosure a fat combination is used including coconut oil, corn oil, olive oil, sunflower oil with high content of oleic acid, palm oil and canola oil, among others. The use of these oils reduces the saturated fat contribution which is harder to absorb and may interfere with calcium absorption, it also allows increasing the monounsaturated fatty acids level until reaching similar concentrations to those of human milk. With these fats combination a quotient between 5 and 15 is reached for linoleic/α-linolenic acid, as well as in human milk this allows favoring the synthesis of polyunsaturated fatty acids of the series n-3.

In the formula the long chain n-6 polyunsaturated fatty acids constitute between 8-35% of the total fatty acids and n-3 from 1 to 5%. Addition of these fatty acids is advisable since they are essential precursors for the eicosanoids and phospholipids synthesis, which are important structural components of the membrane systems, of the central nervous system and the photo-receptor cells of the retina.

The formula of the present disclosure contains docosahexarnoic acid (DHA) in a quantity that represents from 0.2 to 0.7% of the total fatty acids, besides the arachidonic acid (ARA) in ratio from 0.35 to 1.1% of the total fatty acids. These quantities have shown being sufficient in order to obtain a visual acuity which represents the beneficial effect on the development and maturation of the central nervous system.

Another very important component of the lipid fraction of breast milk is the palmitic acid. Palmitic acid represents 25% of the breast milk fatty acids and more than 70% is esterified in the position 2 of the triglyceride. Until recently all infant formulas presented triglycerides with palmitic acid esterified in positions 1 and 3. Esterified palmitic acid in these positions is released during digestion and forms calcium soaps which are insoluble and harden stool. The presence of a higher percentage of beta palmitate has an important effect in the reduction of constipation, colic and other gastrointestinal alterations. In our formulation no less than 45% of the palmitic acid is found as beta palmitate, which reduces in a significant way constipation risk.

In the composition a mixture of vegetal and animal origin triglycerides is used as beta palmitate source which give an esterified palmitic acid in beta position concentration that reaches 45% of the total palmitic acid.

The composition of the present disclosure is found to be enriched with nucleotides in the following proportions, following the same pattern found in breast milk:

Adenosine monophosphate (AMP) 3.2 mg/100 g of formula
Cytidine monophosphate (CMP) 12.0 mg/100 g of formula
Guanosine monophosphate (GMP) 2.5 mg/100 g of formula
Inosine monophosphate (IMP) 1.0 mg/100 g of formula
Uridine monophosphate (UMP) 1.75 g/1 g of formula
The formula in the present disclosure also contains all the vitamins and minerals in the required quantities in order to guarantee an adequate development of the infant. Among the vitamins, minerals and other nutrients which are present in the infant formula are included but not limited to vitamin A, vitamin B1, vitamin B6, vitamin B12, vitamin E, vitamin K, vitamin C, vitamin D, folic acid, inositol, choline, niacin, biotin, pantothenic acid, iron, magnesium, copper, zinc, manganese, chlorine, potassium, sodium, selenium, molybdenum, taurine, L-carnitine. Minerals are added in salt form so they are more stable and of higher bioavailability.

### EXAMPLES

### Example 1

### Feeding Formula for Infants from 0 to 6 Months Old

An example of feeding formula for infants from 0 to 6 months is presented in the following table. As it will be appreciated by the experts on the technique, besides the ingredients specified next the infant formula may also contain other ingredients that are usually included in these particular products, including vitamins and minerals.

| | Quantity per 100 g | |
|---|---|---|
| Nutrient | of powder | Units |
| Energy content | 520 | kcal |
| Protein | Total protein 9.5 | g |
| Whey:Casein 65:35 | | |
| Alpha-lactalbumin 26% of the total | | |
| protein | | |
| <7% de lactoglobuline | | |
| 10% Lactoferrin | | |
| Beta Casein: at least 60% as casein A2. | | |
| Fats (lipids) | 29 | g of which |
| saturated fat | 11 | g |
| polyunsaturated fat | 5.7 | g |
| monounsaturated fat | 10 | g |
| Cholesterol | 0.00 | g |
| 45% of the palmitic acid as beta | | |
| palmitate | | |
| Carbohydrates (Lactose) | 55 | g |
| Sodium | 140.23 | mg |
| Vitamin A | 1800.00 | UI |
| (retinol equivalents) | | |
| Vitamin D | 8.5 | μg |
| (cholecalciferol) | | |
| Vitamin E | 11.00 | mg |
| (tocopherol equivalent) | | |
| Vitamin K | 58.00 | μg |
| Vitamin C | 78.00 | mg |
| (ascorbic acid) | | |
| Vitamin B₁ (thiamine) | 800 | μg |
| Vitamin B₂ (riboflavine) | 900 | μg |
| Vitamin B₆ (piridoxine) | 400 | μg |
| Vitamin B₁₂ (cobalamin) | 2.05 | μg |
| Folic acid (folacin) | 86 | μg |
| Niacin (nicotinic acid equivalent) | 4.5 | Mg |
| Biotin | 17.00 | μg |
| Pantothenic acid | 2.98 | Mg |
| Choline | 80 | Mg |
| Inositol | 70.00 | Mg |
| Potassium | 576.62 | Mg |
| Chloride | 400 | Mg |
| Calcium | 429.70 | Mg |
| Phosphorus | 222.00 | Mg |
| Iron | 7.0 | Mg |
| Magnesium | 52.00 | Mg |
| Zinc | 5.0 | Mg |
| Manganese | 40 | μg |
| Copper | 400.20 | μg |
| Iodine | 80.00 | μg |
| Carnitine¹ | 17 | mg |
| Taurine ² | 40 | mg |
| Linolenic acid³ | 440 | mg |
| Linoleic acid# | 4.1 | g |
| Nucleotides⁵ | 20 | mg |
| DHA | 57 | mg |
| ARA | 98 | mg |
| Selenium⁶ | 17 | mg |
| Fructooligosaccharides (FOS)⁷ | 0.533 | g |
| Galactooligosaccharides (GOS) | 4.79 | g |
| Lutein | 64 | μg |

### Example 2

### Feeding Formula for Infants from 6 to 12 Months Old

An example of feeding formula for infants from 6 to 12 months old is presented on the following table. As it will be appreciated by the experts on the technique, besides the ingredients specified next the infant formula may also contain other ingredients that are usually included in these particular products, including vitamins and minerals.

| | Quantity per 100 g | |
|---|---|---|
| Nutrient | of powder | Units |
| Energy content | 520 | kcal |
| Protein | Total protein 16 | g |
| Whey:Casein 50:50 | | |
| Alpha-lactalbumin 18% of the total | | |
| protein | | |
| <12% de lactoglobulin | | |
| 10% Lactoferrin | | |
| Beta casein: at least 60% as casein A2. | | |
| Fats (lipids) | 25.5 | g of which |
| saturated fat | 8.1 | g |
| polyunsaturated fat | 4.2 | g |
| monounsaturated fat | 7.4 | g |
| trans fatty acids | 140 | mg |
| Cholesterol | 26 | Mg |
| 23% of palmitic acid as beta palmitate | | |
| Carbohydrates (100% Lactose) | 53 | g |
| Sodium | 160 | mg |
| Vitamin A | 1800.00 | UI |
| (retinol equivalents) | | |
| Vitamina D | 10.2 | μg |
| (cholecalciferol) | | |
| Vitamina E | 11.00 | mg |
| (tocopherol equivalent) | | |
| Vitamina K | 50.00 | μg |
| Vitamina C | 78.00 | mg |
| (ascorbic acid) | | |
| Vitamina B₁ (thiamine) | 800 | μg |
| Vitamina B₂ (riboflavine) | 900 | μg |
| Vitamina B₆ (piridoxine) | 450 | μg |
| Vitamina B₁₂ (cobalamin) | 2.05 | μg |
| Ácido fólico (folacin) | 59 | μg |
| Niacina (nicotinic acid equivalent) | 2040 | Mg |
| Biotin | 10.00 | μg |
| Pantothenic acid | 2.98 | Mg |
| Choline | 50 | Mg |
| Inositol | 25 | Mg |
| Potassium | 576.62 | Mg |
| Chloride | 500 | Mg |
| Calcium | 615 | Mg |
| Phosphorus | 350 | Mg |
| Iron | 8.8 | Mg |
| Magnesium | 60.00 | Mg |
| Zinc | 6.0 | Mg |
| Manganese | 257 | μg |
| Copper | 350 | μg |
| Iodine | 100 | μg |
| Carnitine¹ | 17 | mg |
| Taurine ² | 33 | mg |
| Linolenic acid³ | 390 | mg |
| Linoleic acid# | 3.9 | g |
| Nucleotides⁵ | 20 | mg |
| DHA | 26 | mg |
| ARA | 39 | mg |
| Selenium⁶ | 17 | mg |
| Fructooligosaccharides (FOS)⁷ | 0.533 | g |
| Galactooligosaccharides (GOS) | 4.79 | g |
| Lutein | 147 | μg |

### Example 3

### Feeding Formula for Infants from 12 to 36 Months Old

An example of feeding formula for infants from 12 to 36 months old is presented on the following table. As it will be appreciated by the experts on the technique, besides the ingredients specified next the infant formula may also contain other ingredients that are usually included in these particular products, including vitamins and minerals.

| Component | Units | Content/100 g of powder |
|---|---|---|
| Energy | Kcal | 482 |
| Protein (whey 20:casein 80) | g | 12 |
| Alpha lactalbumin 16% of the total | | |
| protein. | | |
| Beta casein 60% as A2 | | |
| Carbohydrates (48% as lactosa) | g | 55 |
| FOS | g | 3 |
| Beta glucan (1,3/1,6) | mg | 21 |
| Fats Total Lipids | g | 21 |
| Saturated fat | g | 3.4 |
| Monounsaturated fat | g | 6.8 |
| Polyunsaturated fat | g | 5.5 |
| Arachidonic acid | mg | 15 |
| Docosahexanoic acid | mg | 40 |
| Linoleic acid | mg | 3977 |
| Linolenic acid | mg | 426 |
| Trans Fats | g | 0.12 |
| Cholesterol | mg | 18 |
| Nucleotides | mg | 20 |
| Taurine | mg | 35 |
| Lutein | μg | 90 |
| Choline | mg | 75 |
| Inositol | mg | 50 |
| L. carnitine | mg | 17 |
| Vitamin A (Retinol) | μg | 405 |
| Vitamin D | UI | 225 |
| Vitamin E (ET) | mg | 6.5 |
| Vitamin K | μg | 42 |
| Vitamin C | mg | 84 |
| Thiamine (Vit B1) | mg | 0.700 |
| Riboflavine (Vit B2) | mg | 1.2 |
| Niacin | mg | 6.0 |
| Vitamin B6 | μg | 520 |
| Folic acid | μg | 130 |
| Pantothenoic acid | mg | 4.0 |
| Vitamin B12 | μg | 1.3 |
| Biotin | μg | 25 |
| Sodium | mg | 210 |
| Potassium | mg | 811 |
| Chloride | mg | 400 |
| Calcium | mg | 570 |
| Phosphorus | mg | 370 |
| Magnesium | mg | 43 |
| Iron | mg | 7.5 |
| Iodine | μg | 130 |
| Copper | mg | 0.33 |
| Zinc | mg | 5.5 |
| Manganese | μg | 50 |
| Selenium | μg | 10 |

The adequate preparation is obtained by mixing 14.5 g of powder with water enough to reach a final volume of 100 mL. A protein concentration of 1.8 g of protein/100 mL is obtained, very similar to that found in breast milk.

The present disclosure has been sufficiently described so that a person with middle knowledge in the matter is able to reproduce and obtain the results mentioned in the present description. Nevertheless, any skillful person in the field of the technique that corresponds to the present disclosure is able to make no descript modifications in the present application. However, if the application of these modifications in a determinate structure or within the process of manufacture requires the material claimed in the following claims, these structures must be content within the range of the disclosure.

1. An infant formula composition for the optimal growth, gastrointestinal protection and immunological protection of infants between 0 and 36 months old, characterized by comprising between 5 and 15 g of protein, preferably 9.5 g, from 40 to 70 g of carbohydrates, preferably 55 g; between 15 to 40 g of fat, preferably 29 g, per every 100 g of the total.

2. The composition of claim 1, characterized by comprising in isolate or combined form: casein; whey milk protein; alpha-lactalbumin; beta-casein A2 protein, lactose; palmitic acid; non-digestible oligosaccharides; long chain polyunsaturated fatty acids (LC PUFAS); nucleotides; vitamins and minerals; inositol; choline; taurine; and carnitine.

3. The composition of claim 1, characterized by providing between 1.5 and 3.0 g of protein/100 mL of reconstituted formula, preferably 1.8 g/100 mL.

4. The composition of claim 2, wherein the whey protein:casein ratio is found between 80:20 and 20:80; preferably 65:35.

5. The composition of claim 2, wherein the alpha lactalbumin content with respect to total protein is found between 20 and 30%, preferably 26%.

6. The composition of claim 2, characterized by out of the total beta casein protein, the ratio between A1 type and A2 type is found between 3:97 and 70:30, preferably in a ratio 50:50.

7. The composition of claim 2, wherein the carbohydrate is selected from the following group: sucrose, lactose, maltodextrin, starch.

8. The composition of claim 2, wherein the carbohydrate is lactose.

9. The composition of claim 2, wherein the palmitic acid is found to form between 10 and 50% of the total fatty acids, preferably 25%.

10. The composition according to claim 2, wherein out of the total palmitic acid, between 25 and 70% is found as esterified in position 2 of glycerol (beta palmitate), preferably 45%.

11. The composition of claim 2, which is enriched with oligosaccharides that are present in breast milk and non-digestible in children and thus have prebiotic activity.

12. The composition according to claim 11, wherein the prebiotic oligosaccharides are fructooligosaccharides and gactooligosaccharides.

13. The composition according to claim 12, wherein the fructooligosaccharides are found in a concentration between 0.05 and 0.2 g per every 100 g of formula; preferably 0.533 g/100 g of formula.

14. The composition according to claim 11, wherein galactooligosaccharides are found in a concentration between 0.1 and 10 g per every 100 g of formula; preferably 4.79 g per every 100 g of formula.

15. The composition of the present disclosure according to claim 2, which is enriched with long chain polyunsaturated fatty acids (LC PUFAS), mainly DHA (docosahexaenoic acid) and ARA (arachidonic acid).

16. The composition according to claim 15, wherein DHA is found in a concentration between 20 and 200 mg per every 100 g of formula, preferably 57 mg/100 g of formula.

17. The composition according to claim 15, wherein ARA is found at a concentration between 20 and 200 mg per every 100 g of formula, preferably 98 mg/100 g of formula.

18. The composition according to claim 2, which is enriched with a nucleotides mixture in a ratio between 5 mg and 50 mg per every 100 grams of powder formula, preferably 24.1 mg/100 g of formula.

19. The composition according to claim 2, wherein the nucleotides mixture is formed as follows:
Adenosine monophosphate (AMP) between 1 mg and 15 mg/100 g of powder formula, preferably 7.45 mg/100 grams of formula.
Cytidine monophosphate (CMP) between 5 and 30 mg/100 g of powder formula, preferably 12.45 mg/100 grams of formula.
Guanosine monophosphate (GMP) between 1 mg and 10 mg per every 100 g of powder formula, preferably 2.48 mg per every 100 grams of formula.
Inosine monophosphate (IMP) between 1 mg and 10 mg per every 100 g of powder formula, preferably 4.97 mg per every 100 grams of formula.
Uridine monophosphate (UMP) between 1 mg and 15 mg per every 100 g of powder formula, preferably 8.6 mg per every 100 grams of formula.

20. A composition according to claim 2, wherein vitamins and minerals are selected among sodium, potassium, calcium, phosphorus, chlorine, magnesium, manganese, iron, copper, zinc, selenium, iodine, vitamin A, C, D, E, K, all vitamins of B complex (B1, B2, B6 and B12), niacin, pantothenic acid and mixes of them.

21. The composition according to claim 2, which is added with inositol in concentrations between 10 and 100 mg per every 100 g of powder, preferably 70 mg per every 100 mg of formula.

22. The composition according to claim 2, which is added with choline in concentrations between 10 and 100 mg per every 100 g of powder, preferably 80 mg per every 100 g of formula.

23. The composition according to claim 2, which is added with taurine in concentrations between 10 and 100 mg per every 100 g of powder, preferably 40 mg per every 100 grams of formula.

24. The composition according to claim 2, which is added with carnitine in concentrations between 1 and 50 mg per every 100 g of powder, preferably 17 mg per every 100 grams of formula.

25. The composition according to claim 2, wherein the final product may be powder which must be mixed with water before its use or wherein the final product may be presented as a sterile liquid ready to use.

26. The composition of the infant formulas according to claims 1 to 25 wherein the products are presented as powders to reconstitute, sterile liquids ready to use or any other form in which children of any age or adults may ingest them.

27. An infant formula composition for the optimal growth, gastrointestinal protection and immunological protection of infants between 0 and 6 months of life characterized by comprising at least 60% of beta casein A2 out of the total beta casein protein.

28. An infant formula composition for the optimal growth, gastrointestinal protection and immunological protection of infants between 6 and 12 months of life characterized by comprising at least 60% of beta casein A2 out of the total beta casein protein.

29. An infant formula composition for the optimal growth, gastrointestinal protection and immunological protection of infants between 12 and 36 months of life characterized by comprising at least 60% of beta casein A2 out of the total beta casein protein.

30. The use of beta casein A2, for preparing an infant formula composition for the optimal growth, gastrointestinal protection and immunological protection of infants wherein out of the total beta casein protein, the ratio between A1 and A2 type is between 3:97 and 70:30, preferably in a ratio 50:50.
